(19) United States
(12) Patent Application Publication
(10) Pub. No.: US 2018/0370893 A1
(43) Pub. Date: Dec. 27, 2018

(54) NEW METHOD FOR SYNTHESIZING 2-FLUOROCYCLOPROPANE CARBOXYLIC ACID

(71) Applicant: CHEN-STONE (GUANGZHOU) CO., LTD., Guangzhou, GD (CN)

(72) Inventors: Yingjie LAI, Guangzhou (CN); Xuyan WANG, Guangzhou (CN)

(21) Appl. No.: 16/061,880

(22) PCT Filed: Apr. 25, 2017
(86) PCT No.: PCT/CN2017/081892

§ 371 Date: Jun. 13, 2018

(30) Foreign Application Priority Data
Aug. 18, 2016 (CN) 201610686205.9

(51) Int. Cl. C07C 51/09; C07C 61/15

(52) U.S. Cl. CPC C07C 51/09; C07C 317/22; C07C 61/15

(57) ABSTRACT

Disclosed is a new method for synthesizing 2-fluorocyclopropanecarboxylic acid comprising: 1) performing reaction of 1,1-dichloro-1-fluoroethane with thiophenol in the presence of an alkali, to produce a phenyl sulfide intermediate; 2) performing oxidation reaction of the phenyl sulfide intermediate with Oxone; 3) performing elimination reaction of the product of Step 2) in the presence of an alkali, to obtain 1-fluoro-1-benzenesulfonyl ethylene; 4) performing addition reaction of the 1-fluoro-benzenesulfonyl ethylene with ethyl diazoacetate in the presence of a catalyst, to obtain a cyclopropane intermediate; 5) performing elimination reaction of the cyclopropane intermediate in the presence of an alkali before acidification, to obtain 2-fluorocyclopropanecarboxylic acid. Herein, the synthetic route is short, used materials are bulk commodities, and raw materials are inexpensive and readily available. The process can be safely scaled up by replacing commonly used mCPBA reagents with Oxone. Further, reaction yield is improved, production cost is greatly reduced, and operation is simplified.

### FIELD OF THE INVENTION

The present disclosure relates to a new method for synthesizing 2-fluorocyclopropanecarboxylic acid.

### BACKGROUND OF THE INVENTION

Since a fluorine atom has the largest electronegativity and oxidation potential, the introduction of a fluorine atom into a drug molecule can increase the lipophilicity of the drug and improve the trans-membrane ability of the drug in a living body, without significant alteration of the volume of the drug molecular. Thus, the bioavailability of the drug would be increased. In 1954, Fried and Sabo discovered that 9a-fluoroacetic acid cortisone prepared by introducing a fluorine atom into cortisone acetate exhibited an anti-inflammatory effect about 15 times higher than that of hydrocortisone. For the first time, the fluorine atom was proved to be efficient for increasing the biological activity of a drug. With the development of fluorine chemistry, fluorine atoms are included by increasing drug molecules, such as atorvastatin calcium, levofloxacin, lansoprazole, efavirenz, ezetimibe and so on.

In recent ten to twenty years, a fluorocyclopropane structural unit has been one of hot topics in the international research of fluorinated drug molecules. Increasing bioactive molecules containing fluorocyclopropane structures have been discovered and some of them have entered into clinical research.

Sitafloxacin, as a new broad-spectrum quinolone-based antibacterial drug, has appeared on the market in Japan and will further come into the market in various countries such as China and South Korea in recent years. It has a very broad market prospect. One of the side chains of sitafloxacin is monofluorocyclopropane. The synthesis of this fragment requires a key intermediate (1S, 2S)-2-fluorocyclopropane carboxylic acid. However, it is difficult and high cost for the synthesis of 2-fluorocyclopropane carboxylic acid. As a result, the raw material of sitafloxacin is expensive, which may impair the market promotion thereof. Therefore, it is necessary to develop a novel, highly effective and cost-effective technology for the synthesis of 2-fluorocyclopropane carboxylic acid.

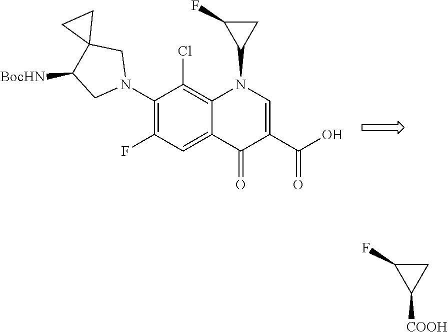

### Sitafloxacin (1S, 2S)-2-fluorocyclopropane carboxylic acid

Now, there are several methods for synthesizing 2-fluorocyclopropane carboxylic acid:

Regarding Method I, carbene is prepared by using polyhalogenated alkane and a cyclopropane intermediate is obtained by a one-pot process. In the method published by Bayer Pharmaceuticals in 1990, butadiene was used as a starting material and a remaining alkenyl group on the resulted cyclopropane intermediate was oxidized, thereby obtaining 2-fluorocyclopropane carboxylic acid (J. of Fluorine chem., 1990, 49, 127).

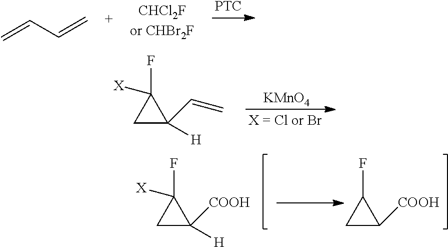

If this method uses low-cost dichlorofluoromethane as a starting material, it would be difficult to generate carbene due to the low activity of dichlorofluoromethane, and thus the yield of cyclopropylation reaction would be low (31%). If expensive dibromofluoromethane is used as a starting material, the cost would be extremely high due to the low atomic utilization ratio thereof. With literature research, it is found that dichlorofluoromethane performs a carbene addition reaction, the yield of which is generally low, under an alkaline condition with a phase transfer catalyst. As reported by Sauers's group from University of New Jersey, the yield of 1-fluoro-1-chloro-2, 2-dimethylcyclopropane was only 8% by a reaction of dichlorofluoromethane with isobutylene (J. Am. Chem. Soc. 2005, 127, 2408). Furthermore, it was reported by Craig's group from Duke University that the yield of a reaction of dichlorofluoromethane with cyclooctadiene was only 35% (J. Am. Chem. Soc. 2015, 137, 11554).

Method II is a method developed by Daiichi Sankyo Pharmaceutical Co. Ltd. in 1995. In this method, Freon was reacted with thiophenol, and then the resulted phenyl sulfide was reacted with t-butyl acrylate to obtain a corresponding cyclopropane intermediate (JPH0717945).

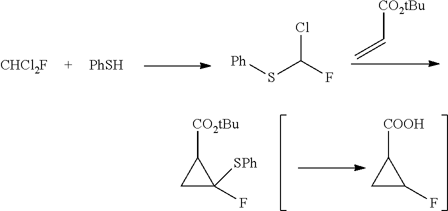

This method requires high concentration of potassium hydroxide and sodium hydroxide solutions, heating, has high requirements for the equipment, and produces a large amount of process wastewater, which is detrimental to environmental protection. Moreover, a violent reaction condition results in many side reactions, and the product must be separated through rectification. However, it is difficult to achieve the rectification in a factory, due to the high boiling point of the product.

Method III is Michael addition of t-butyl acrylate, which was developed by Daiichi Sankyo Pharmaceutical Co. Ltd. in 1996 (Tetrahedron Lett. 1996, 47, 8507). This reaction was carried out at an ultra-low temperature and NaHMDS was used as an alkali, resulting in a yield of 51%. Then, the resulted intermediate sulfoxide was reacted with fluorine gas to produce a 2-fluoro intermediate.

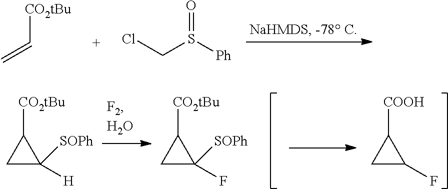

In this method, an ultra-low temperature reaction is performed in the first step, which has high requirements for the equipment and is highly cost. Further, fluorine gas is used in the second step. Because of the strong corrosivity and oxidability of fluorine gas, there are many problems in operability and safety. Thus, this method is not suitable for industrial production.

Method IV is a cycloaddition reaction of ethyl diazoacetate with fluoroolefin. The addition reaction of carbene with a carbon-carbon double bond is one of classical methods for the synthesis of cyclopropane. Patent WO20100005003, which was published by Daiichi Sankyo Pharmaceutical Co. Ltd. in 2009, cites that an asymmetric copper catalyst is used to catalyze the cycloaddition reaction of 1,1-fluorochloroethylene with ethyl diazoacetate.

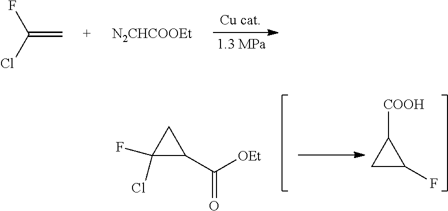

This method is a relatively classical method. However, this method uses 1, 1-fluorochloroolefin in a form of gas, which is easily escaped when releasing nitrogen during the reaction. Thus, 1, 1-fluorochloroolefin should be greatly excessive, and the process is unstable. Moreover, this reaction is required to be performed under a closed condition, which leads to greater security risk in production.

Method V is a rhodium-catalyzed method which was developed by Kyorin Pharmaceutical Co., Ltd. (Japan) in 2014. This method was based on method II, and used 1-fluoro-1-phenylsulfone ethylene, instead of 1, 1-fluorochloroalkene, to carry out the carbene reaction. In the resulted intermediate, a trans/cis ratio reached 86/14, which greatly enhanced cis/trans selectivity.

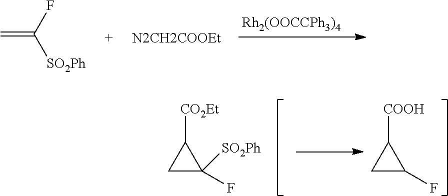

This method uses 1-fluoro-1-benzenesulfonyl ethylene and avoids the use of 1,1-fluorochloroolefin. Although the gas escape problem in method IV above is avoided, it is difficult and costly for the preparation of the 1-fluoro-1-benzenesulfonyl ethylene (the synthesis route thereof shown as follows).

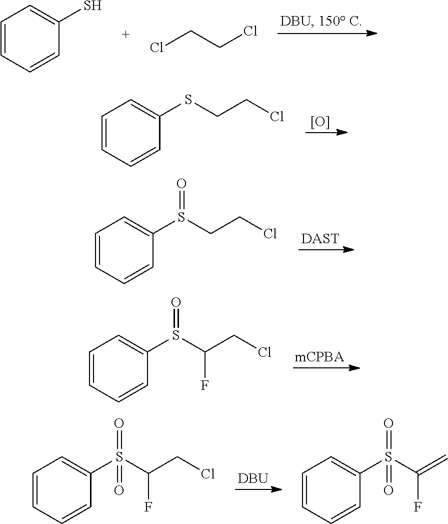

All of the above methods have a disadvantage of being difficult to scale up. The manufacturers of 2-fluorocyclopropane carboxylic acid are quite few, and 2-fluorocyclopropane carboxylic acid is extremely expensive, which seriously impedes further application and development thereof in organic chemistry and biomedicine. Therefore, there will be greatly practical benefit to develop a process that can be safely scaled up.

### SUMMARY OF THE INVENTION

The object of the present disclosure is to provide a new method for synthesizing 2-fluorocyclopropane carboxylic acid.

The technical solution of the present disclosure is as follows:

A new method for synthesizing 2-fluorocyclopropanecarboxylic acid comprises the following steps:

1) performing a reaction of 1,1-dichloro-1-fluoroethane with thiophenol in the presence of an alkali, to produce a phenyl sulfide intermediate;

2) performing an oxidation reaction of the phenyl sulfide intermediate with Oxone;

3) performing an elimination reaction of the product obtained in Step 2) in the presence of an alkali, to obtain 1-fluoro-1-benzenesulfonyl ethylene;

4) performing an addition reaction of 1-fluoro-1-benzenesulfonyl ethylene with ethyl diazoacetate in the presence of a catalyst, to obtain a cyclopropane intermediate;

5) performing an elimination reaction of the cyclopropane intermediate in the presence of an alkali before an acidification, to obtain 2-fluorocyclopropanecarboxylic acid.

In Step 1), the alkali is at least one selected from alkoxide, carbonate, bicarbonate, hydroxide, and hydride of an alkali metal or alkaline earth metal.

In Step 1), the mass ratio of 1,1-dichloro-1-fluoroethane and thiophenol is (1.1-3.5):1.

In Step 2), the mass ratio of the phenyl sulfide intermediate and Oxone is 1:(7-9).

In Step 3), the alkali is at least one selected from alkoxide, carbonate, bicarbonate, hydroxide and hydride of an alkali metal or alkaline earth metal and DBU.

In Step 3), the mass ratio of the product obtained in Step 2) and the alkali is (1.1-2):1.

In Step 4), the mass ratio of 1-fluoro-1-benzenesulfonylethylene and ethyl diazoacetate is (1.1-1.7):1.

In Step 4), the catalyst is a rhodium-based catalyst.

In Step 5), the alkali is at least one selected from alkoxide, carbonate, bicarbonate, hydroxide, hydride of an alkali metal or alkaline earth metal.

In Step 5), an acid for the acidification is at least one selected from hydrochloric acid, sulfuric acid, nitric acid and perchloric acid.

The advantages of the present disclosure are that:

1. The synthetic route of the present disclosure is short, the materials used therein are bulk commodities, and the raw materials are inexpensive and readily available.

2. The process can be safely scaled up by replacing commonly used mCPBA reagents with Oxone.

3. The reaction yield is increased, the production cost is greatly reduced, and the operation is simplified.

### BRIEF DESCRIPTION OF THE DRAWINGS

FIG. 1 is a schematic diagram of a synthesis method according to the present disclosure.

### DETAILED DESCRIPTION OF THE EMBODIMENTS

A new method for synthesizing 2-fluorocyclopropane carboxylic acid comprises the following steps:

1) performing a reaction of 1,1-dichloro-1-fluoroethane with thiophenol in the presence of an alkali, to produce a phenyl sulfide intermediate;

2) performing an oxidation reaction of the phenyl sulfide intermediate with Oxone;

3) performing an elimination reaction of the product obtained in Step 2) in the presence of an alkali, to obtain 1-fluoro-1-benzenesulfonyl ethylene;

4) performing an addition reaction of 1-fluoro-1-benzenesulfonyl ethylene with ethyl diazoacetate in the presence of a catalyst, to obtain a cyclopropane intermediate;

5) performing an elimination reaction of the cyclopropane intermediate in the presence of an alkali before an acidification, to obtain 2-fluorocyclopropanecarboxylic acid.

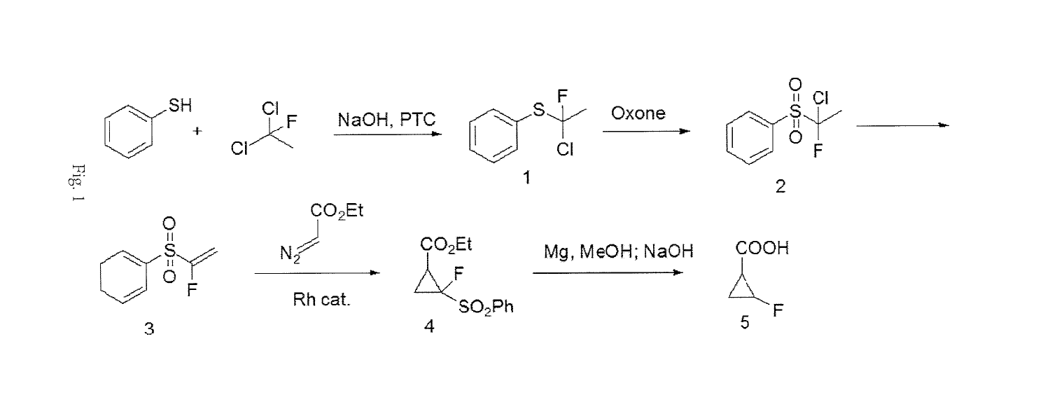

FIG. 1 is a schematic diagram of the synthesizing method according to the present disclosure. The schematic diagram is merely an example of the synthesizing method. It shall be appreciated that the method of the present disclosure is not limited to the related materials as shown in FIG. 1.

Preferably, the alkali of Step 1) is at least one selected from alkoxide, carbonate, bicarbonate, hydroxide, and hydride of an alkali metal or alkaline earth metal. More preferably, the alkali of Step 1) is at least one selected from sodium alkoxide, potassium alkoxide, sodium hydroxide, potassium hydroxide, sodium hydride, potassium hydride, sodium carbonate, potassium carbonate, sodium bicarbonate, and potassium bicarbonate. Still more preferably, the alkali of Step 1) is at least one selected from sodium alkoxide, potassium alkoxide, sodium hydroxide and potassium hydroxide. Still more preferably, the alkali of Step 1) is at least one selected from sodium hydroxide and potassium hydroxide.

Preferably, in Step 1), the mass ratio of 1,1-dichloro-1-fluoroethane and thiophenol is (1.1-3.5):1. More preferably, in Step 1), the mass ratio of 1,1-bischloro-1-fluoroethane and thiophenol is (1.2-3.4):1. Still more preferably, in Step 1), the mass ratio of 1,1-dichloro-1-fluoroethane and thiophenol is (1.3-3.3):1.

Preferably, in Step 2), the mass ratio of the phenyl sulfide intermediate and Oxone is 1: (7-9). More preferably, in Step 2), the mass ratio of the phenyl sulfide intermediate and Oxone is 1: (7.2-8.8). More preferably, in Step 2), the mass ratio of the phenyl sulfide intermediate and Oxone is 1:(7.4-8.6). Still more preferably, in Step 2), the mass ratio of the phenyl sulfide intermediate and Oxone is 1: (7.6-8.4).

Preferably, the alkali of Step 3) is at least one selected from alkoxide, carbonate, bicarbonate, hydroxide, and hydride of an alkali metal or alkaline earth metal and DBU. More preferably, the alkali of Step 3) is at least one selected from sodium alkoxide, potassium alkoxide, sodium hydroxide, potassium hydroxide, sodium hydride, potassium hydride, sodium carbonate, potassium carbonate, sodium bicarbonate, potassium bicarbonate, and DBU. More preferably, the alkali of Step 3) is at least one selected from sodium alkoxide, potassium alkoxide, sodium hydroxide, potassium hydroxide, and DBU. Still more preferably, the alkali of Step 3) is at least one selected from potassium t-butoxide, potassium hydroxide, and DBU.

Preferably, in step 3), the mass ratio of the product obtained in step 2) and the alkali is (1.1-2):1. More preferably, in step 3), the mass ratio of the product obtained in Step 2) and the alkali is (1.2-1.9):1. Still more preferably, in step 3), the mass ratio of the product obtained in Step 2) and the alkali is (1.3-1.8):1.

Preferably, the solvent for the reaction of Step 3) is a polar solvent. More preferably, the solvent for the reaction of Step 3) is at least one selected from water, methanol, ethanol, propanol, isopropanol, acetone, tetrahydrofuran, dimethyl sulfoxide. Still more preferably, the solvent for the reaction of Step 3) is at least one selected from water, methanol, and tetrahydrofuran.

Preferably, in Step 4), the mass ratio of 1-fluoro-1-benzenesulfonylethylene and ethyl diazoacetate is (1.1-1.7):1. More preferably, in Step 4), the mass ratio of 1-fluoro-1-benzenesulfonyl ethylene and ethyl diazoacetate is (1.2-1.6):1. Still more preferably, in Step 4), the mass ratio of 1-fluoro-1-benzenesulfonylethylene and ethyl diazoacetate is (1.3-1.5):1.

Preferably, the catalyst of Step 4) is a rhodium-based catalyst. More preferably, the catalyst of Step 4) is an organic rhodium catalyst. Still more preferably, the catalyst of Step 4) is a rhodium acetate dimer. Most preferably, the catalyst of Step 4) is a rhodium triphenylacetate dimer.

Preferably, in Step 4), the mass ratio of the catalyst to 1-fluoro-1-phenylsulfonylethylene is 0.5-1.5%. More preferably, in Step 4), the mass ratio of the catalyst to 1-fluoro-1-phenylsulfonylethylene is 0.8-1.2%. Most preferably, in step 4), the mass ratio of the catalyst to 1-fluoro-1-benzenesulfonylethylene is 1.0%.

Preferably, the alkali of Step 5) is at least one selected from alkoxide, carbonate, bicarbonate, hydroxide, and hydride of an alkali metal or alkaline earth metal. More preferably, the alkali of Step 5) is at least one selected from sodium alkoxide, potassium alkoxide, magnesium alkoxide, sodium carbonate, potassium carbonate, sodium bicarbonate, potassium bicarbonate, sodium hydroxide, potassium hydroxide, sodium hydride, potassium hydride. Still more preferably, the alkali of Step 5) is at least one selected from magnesium ethoxide, sodium ethoxide, potassium t-butoxide, sodium hydroxide, and potassium hydroxide. Still more preferably, the alkali of Step 5) is at least one selected from magnesium ethoxide, sodium hydroxide and potassium hydroxide.

Preferably, in step 5), the acid for the acidification is at least one selected from hydrochloric acid, sulfuric acid, nitric acid and perchloric acid. More preferably, in step 5), the acid for the acidification is at least one selected from hydrochloric acid and sulfuric acid. Most preferably, in step 5), the acid for the acidification is hydrochloric acid.

Hereinafter the contents of the present disclosure will be explained in further details with reference to specific examples.

### EXAMPLE

### Example 1 for Step 1

At room temperature, 10 g of thiophenol was added into 50 mL of methanol, and then 18 g of 40% NaOH solution was gradually added. 32 g of precooled 1, 1-dichloro-1-fluoroethane (Freon 141b) was added into the mixture, followed by stirring intensely overnight at 40-50° C. The reaction solution was cooled to room temperature, and 20 mL of concentrated hydrochloric acid was gradually added. The reaction solution was concentrated, so as to remove most of methanol. Then, it was extracted with ethyl acetate, washed with a saturated sodium carbonate solution, dried, and concentrated, so as to obtain 11 g of a crude product of a phenyl sulfide intermediate (Product 1 as shown in FIG. 1). The yield of the crude product was 59%.

### Example 2 for Step 1

In an ice bath, 15 g of thiophenol, 20 g of 1,1-dichloro-1-fluoroethane, and 1.5 g of triethyl benzyl ammonium chloride were added into 100 mL of a toluene solution. After stirring, 80 mL of 50% sodium hydroxide solution was gradually added, followed by stirring intensely overnight at room temperature. The reaction solution was extracted twice with toluene. The separated organic phase was washed with saturated sodium bicarbonate, dried, and concentrated, so as to obtain 20 g of a crude product of a phenylene sulfide intermediate (Product 1 as shown in FIG. 1). The yield of the crude product was 71%.

### Example 1 for Step 2

117 g of Oxone was added into 175 mL water at room temperature. The reaction solution was cooled to 0° C., and then 14 g of the crude phenyl sulfide intermediate in methanol (175 mL) was gradually added. The temperature of the reaction solution was slowly increased to room temperature, followed by stirring overnight. It was concentrated so as to remove methanol. The reaction solution was extracted twice with 200 mL of dichloromethane. The organic phase was washed with saturated brine, dried and concentrated to give 18 g of a yellow oily product (Product 2 as shown in FIG. 1).

### Example 2 for Step 2

115 g of Oxone was added into 85 mL water at room temperature. The reaction solution was cooled to 0° C. and then 15 g of the crude phenyl sulfide intermediate in methanol (85 mL) was gradually added. The temperature of the reaction solution was slowly increased to room temperature, followed by stirring overnight. The reaction solution was filtered with diatomite. The filtrate was concentrated to remove methanol, and then the reaction solution was extracted twice with 200 mL of dichloromethane. The organic phase was washed with brine, dried and concentrated to give 16 g of a yellow oily product (Product 2 as shown in FIG. 1).

### Example 1 for Step 3

11 g of potassium t-butoxide was dissolved in 100 mL of THF and cooled to 0° C. 15 g of the yellow oily product obtained in Step 2) was dissolved in 50 mL of THF and gradually added dropwise into the potassium t-butoxide solution. The temperature of the reaction solution was slowly increased to room temperature, followed by heating reflux overnight. After cooling, 200 mL of a saturated ammonium chloride solution was added and then concentrated to remove a part of THF. The reaction solution was extracted twice with ethyl acetate. The organic phase was washed with saturated sodium bicarbonate, dried, concentrated, followed by crystallization with n-hexane, so as to give 12 g of a light brown solid (Product 3 as shown in FIG. 1).

### Example 2 for Step 3

16 g of potassium hydroxide was dissolved in 12 mL of water and stirred for 0.5 h. Then, 12 g of methanol was gradually added. After stirring, 26 g of the yellow oily product obtained in Step 2) was added into the reaction solution. Then, the reaction solution was heated to 90° C. and hold for 3 hours, and then was cooled to room temperature. The reaction solution was extracted three times with methyl t-butyl ether. The organic phase was washed with saturated brine, dried, and concentrated, followed by crystallization with n-hexane, so as to give 18 g of a light brown solid (Product 3 as shown in FIG. 1).

### Example for Step 4

17 g of the product obtained in Step 3) and 0.17 g of a rhodium triphenylacetate dimer catalyst were dissolved in 50 mL of methylene chloride, and then 12 g of ethyl diazoacetate in dichloromethane (40 mL) was gradually added dropwise. After stirring for 2 hours, the reaction solution was washed with diluted hydrochloric acid and washed with saturated sodium bicarbonate solution. The organic phase was concentrated to give 35 g of an oily product (Product 4 as shown in FIG. 1).

### Example for Step 5

The oily product obtained in Step 4) was dissolved in 50 mL of ethanol and then 6.5 g of magnesium powder and 1 g of mercuric chloride were added. The mixture was stirred overnight and then poured into 50 mL of diluted hydrochloric acid (IN). It was extracted three times with n-hexane, and then the organic phase was dried, filtered and concentrated. The concentrated crude product was added into a solution of 30 mL water and 4 g sodium hydroxide, and stirred for one hour. The reaction solution was acidified with concentrated hydrochloric acid to realize pH=1. It was extracted three times with methyl t-butyl ether. The combined organic phases were concentrated, and then 10 mL of isopropyl ether was added, cooled and crystallized, so as to give 6.1 g of a white solid of 2-fluorocyclopropanecarboxylic acid (Product 5 as shown in FIG. 1).

1: A new method for synthesizing 2-fluorocyclopropanecarboxylic acid,
comprising the following steps:
1) performing a reaction of 1,1-dichloro-1-fluoroethane with thiophenol in the presence of an alkali, to produce a phenyl sulfide intermediate;
2) performing an oxidation reaction of the phenyl sulfide intermediate with Oxone;
3) performing an elimination reaction of a product obtained in Step 2) in the presence of an alkali, to obtain 1-fluoro-1-benzenesulfonyl ethylene;
4) performing an addition reaction of the 1-fluoro-1-benzenesulfonyl ethylene and ethyl diazoacetate in the presence of a catalyst, to obtain a cyclopropane intermediate;
5) performing an elimination reaction of the cyclopropane intermediate in the presence of an alkali before an acidification, to obtain 2-fluorocyclopropanecarboxylic acid.

2: The new method for synthesizing 2-fluorocyclopropanecarboxylic acid of claim 1, wherein, the alkali of Step 1) is at least one selected from alkoxide, carbonate, bicarbonate, hydroxide, and hydride of an alkali metal or alkaline earth metal.

3: The new method for synthesizing 2-fluorocyclopropanecarboxylic acid of claim 2, wherein, in the step 1), the mass ratio of 1,1-dichloro-1-fluoroethane and thiophenol is (1.1-3.5):1.

4: The new method for synthesizing 2-fluorocyclopropanecarboxylic acid of claim 1, wherein, in the step 2), the mass ratio of the phenyl sulfide intermediate and Oxone is 1:(7-9).

5: The new method for synthesizing 2-fluorocyclopropanecarboxylic acid of claim 1, wherein, the alkali of Step 3) is at least one selected from alkoxide, carbonate, bicarbonate, hydroxide and hydride of an alkali metal or alkaline earth metal, and DBU.

6: The new method for synthesizing 2-fluorocyclopropanecarboxylic acid of claim 5, wherein, in Step 3), the mass ratio of the product obtained in step 2) and the alkali is (1.1-2):1.

7: The new method for synthesizing 2-fluorocyclopropanecarboxylic acid of claim 1, wherein, in Step 4), the mass ratio of 1-fluoro-1-benzenesulfonylethylene and ethyl diazoacetate is (1.1-1.7):1.

8: The new method for synthesizing 2-fluorocyclopropanecarboxylic acid of claim 1, wherein, the catalyst of Step 4) is a rhodium-based catalyst.

9: The new method for synthesizing 2-fluorocyclopropanecarboxylic acid of claim 1, wherein, the alkali of Step 5) is at least one selected from alkoxide, carbonate, bicarbonate, hydroxide, and hydride of an alkali metal or alkaline earth metal.

10: The new method for synthesizing 2-fluorocyclopropanecarboxylic acid of claim 9, wherein, in the step 5), an acid for the acidification is at least one selected from hydrochloric acid, sulfuric acid, nitric acid and perchloric acid.
